(19) United States
(12) Patent Application Publication
(10) Pub. No.: US 2013/0078313 A1
(43) Pub. Date: Mar. 28, 2013

(54) MILK DERIVED COMPOSITION AND USE TO ENHANCE MUSCLE MASS OR MUSCLE STRENGTH

(71) Applicants: Michelle ROWNEY, Port Campbell (AU); David CAMERON-SMITH, Ashburton (AU); Scott CONNELLY, Corona Del Mar, CA (US)

(72) Inventors: Michelle ROWNEY, Port Campbell (AU); David CAMERON-SMITH, Ashburton (AU); Scott CONNELLY, Corona Del Mar, CA (US)

(73) Assignee: MURRAY GOULBURN CO-OPERATIVE CO LIMITED, Brunswick (AU)

(21) Appl. No.: 13/479,665

(22) Filed: May 24, 2012

Related U.S. Application Data
(63) Continuation of application No. 11/991,669, filed on Feb. 6, 2009, now abandoned.

(30) Foreign Application Priority Data
Sep. 9, 2005 (AU) 2005904981

(51) Int. Cl. A61K 35/20

(52) U.S. Cl. CPC A61K 35/20
USPC 424/535

(57) ABSTRACT

The invention relates to the production of compositions containing milk products for use as a nutritional supplement. More specifically, it relates to compositions containing whey growth factor extract, as well as methods for supplementing the nutritional needs of individuals undertaking resistance exercise training. According to one aspect of the invention, there is provided the use of a composition comprising whey growth factor extract, isolated from a milk product by cation exchange chromatography, to increase skeletal muscle strength.

### FIELD OF THE INVENTION

The invention relates to the production of compositions containing milk products for use as a nutritional supplement. More specifically, it relates to compositions containing whey growth factor extract, as well as methods for supplementing the nutritional needs of individuals undertaking resistance exercise training.

### BACKGROUND OF THE INVENTION

The present invention is to be understood in light of what has previously been done in the field. However, the following discussion is not an acknowledgement or admission that any of the material referred to was published, used or part of the common general knowledge in Australia as at the priority date of the application.

The use of nutritional supplements by humans, and even animals, to improve general health or to improve, for example, athletic performance, is known. Nutritional supplements are not intended to provide all the nutrients necessary for a complete diet, but instead are generally intended to complement the dietary intake such that it becomes more nutritionally complete. It is recognised that vitamins, minerals and other substances found in such supplements play important physiological roles and that a deficiency of certain vitamins, minerals and/or other components of supplements has been linked to development of certain diseases, a decrease in general health or lower performance in athletes.

Conversely, nutritional supplements are known to enhance a variety of physiological states, under various conditions. There are many targets for nutritional supplements, for example sick patients, convalescing patients, elderly persons and persons undergoing strenuous exercise regimes who wish to improve their performance and/or recovery from such exercise.

The nutritional requirements of bodybuilders and persons engaged in strenuous physical exercise are quite particular, whether to decrease body fat and increase lean muscle mass/size or strength, to improve speed and/or endurance, and/or to improve recovery from the strenuous exercise. Protein supplementation has been used widely by the aforementioned group of persons to promote muscle protein synthesis in order to repair muscle tissue and facilitate muscle growth. The nutritional supplementation may be provided in the form of a drink or food and includes protein powders to be mixed with liquid for use, nutritional bars and snack foods, tablets, capsules and other preparations.

Suitable protein sources commercially available include hydrolysed milk proteins, caseinates, soy protein isolates and milk protein concentrates prepared from ultra-filtrated skim milk. Nutritional supplements which are based on other protein sources, such as whey protein, are also available and can be provided in the form of fruit juices, but are thought to be inadequate because they do not also provide a lipid source (WO 02/15720). In addition, it has been considered that some milk-derived proteins are not readily absorbed by the gut, or do not survive the harsh environment of the digestive system to have a therapeutic effect. Whey growth factor extract is one such milk product which was hitherto not thought to be useful as a nutritional supplement because any biologically active proteins within the extract would be expected to lose activity once ingested.

Nevertheless, the capacity of whey protein supplementation to provide beneficial gains in muscular mass/size and strength in persons engaged in resistance exercise training has been reported to provide a benefit. Whey protein isolate (WPI) and milk protein isolate (MPI) have been reported to be effective with bodybuilders in rapidly gaining lean muscle mass/size while reducing body fat. WPI is high in branched-chain amino acids and considered to be ‘fast’ acting whereas MPI is mainly casein which is more slowly metabolised and is effective in promoting muscle growth. Egg albumin is an alternative to provide a high quality amino acid source.

Recent data provides evidence that supplementation by way of proteins either before and/or after exercise is able to stimulate greater protein synthesis, although the gains in muscle mass are small and variable (Andersen et al, Metabolism, 2005, 54(2):151-156).

The present inventors have discovered that a composition comprising whey growth factor extract significantly increases gains in muscle strength over that achieved by prior art compositions, including a composition comprising WPI alone.

### SUMMARY OF THE INVENTION

The invention relates to a composition and method which enables persons undergoing resistance exercise training to further increase their muscle strength.

It is thus an object of the present invention to provide a composition and method which is improved over the prior art for increasing muscle strength.

According to one aspect of the invention, there is provided a skeletal muscle strength-enhancing composition comprising whey growth factor extract, isolated from a milk product by cation exchange chromatography.

According to another aspect of the invention, there is provided a skeletal muscle strength-enhancing composition comprising whey growth factor extract, isolated from a milk product by a process comprising the steps;
a) applying the milk product to a SP Sepharose cation exchange column,
b) washing the column with a buffer of low ionic strength,
c) eluting the WGFE fraction with a buffer containing in the range 0.4-0.5M NaCl, or equivalent ionic strength, at pH 6.5.

According to another aspect of the invention, there is provided a skeletal muscle strength-enhancing composition comprising whey growth factor extract, isolated from a milk product by a process comprising the steps;
a) applying the milk product to a SP Sepharose cation exchange column,
b) washing the column with a buffer of 0.008M NaCl or less,
c) eluting the WGFE fraction with a buffer containing 0.4M NaCl, or equivalent ionic strength, at pH 6.5.

In a further aspect of the invention, there is provided a composition according to the above wherein the whey growth factor extract is isolated from a milk product selected from whole milk, cheese whey, rennet casein whey, acid casein whey, or concentrates thereof, or skim milk.

In a further aspect of the invention, there is provided a composition according to the above when used as a muscle strength and/or size enhancing agent.

In a further aspect of the invention there is provided a method of improving skeletal muscle strength and/or size in subjects undertaking resistance exercise training comprising administering to the subjects an efficacious amount of a composition comprising whey growth factor extract, isolated from a milk product by cation exchange chromatography.

In a further aspect of the invention there is provided a method of improving skeletal muscle strength and/or size in subjects undertaking resistance exercise training comprising administering to the subjects an efficacious amount of a composition comprising whey growth factor extract, isolated from a milk product by a process comprising the steps;
a) applying the milk product to a SP Sepharose cation exchange column,
b) washing the column with a buffer of low ionic strength,
c) eluting the WGFE fraction with a buffer containing in the range 0.4-0.5M NaCl, or equivalent ionic strength, at pH 6.5.

In a further aspect of the invention there is provided a method of improving skeletal muscle strength and/or size in subjects undertaking resistance exercise training comprising administering to the subjects an efficacious amount of a composition comprising whey growth factor extract, isolated from a milk product by a process comprising the steps;
a) applying the milk product to a SP Sepharose cation exchange column,
b) washing the column with a buffer of 0.008M NaCl or less,
c) eluting the WGFE fraction with a buffer containing 0.4M NaCl, or equivalent ionic strength, at pH 6.5.

In another aspect of the invention there is provided a a method of improving skeletal muscle strength and/or size in subjects undertaking resistance exercise training, wherein the amount of whey growth factor extract administered, per daily dose, is at least 5 mg/kg body weight to 12.5 mg/kg body weight. Preferably the daily dose of whey growth factor extract is at least 25 mg/kg body weight.

In a further aspect of the invention there is provided a use of whey growth factor extract, isolated from a milk product by cation exchange chromatography, for the production of a medicament for the treatment of a subject in need of improved skeletal muscle strength and/or size.

In a further aspect of the invention there is provided a use of whey growth factor extract, isolated from a milk product by a process described above, for the production of a medicament for the treatment of a subject in need of improved skeletal muscle strength and/or size.

According to another aspect of the invention, there is provided a skeletal muscle strength-enhancing composition comprising whey growth factor extract, isolated from a milk product by cation exchange chromatography, and an additional protein source.

According to a further aspect of the invention the additional protein source is whey protein, preferably whey protein isolate (WPI), more preferably whey protein isolate wherein the whey protein isolate comprises:

| | |
|---|---|
| Moisture | 5.0% |
| Fat | 0.5% |
| pH (5% solution) | 6.3 |
| Ash | 3.7% |
| Lactose | 0.5% |
| Protein (TN × 6.38) | 90.0% |
| Sodium | 0.7% |
| Phosphorous | 0.3% |
| Calcium | 0.15% |

In a further aspect of the invention there is provided a method of increasing skeletal muscle strength and/or size in a subject undertaking resistance exercise training comprising administering to the subject an efficacious amount of a composition comprising whey growth factor extract and an additional protein source, such as whey, preferably WPI.

In a further aspect of the invention there is provided a method of increasing skeletal muscle strength and/or size in a subject undertaking resistance exercise training comprising administering to the subject a composition comprising an effective amount of whey growth factor extract, isolated from a milk product by cation exchange chromatography, and an additional protein source taken separately.

Preferably the amount of additional protein source administered, per daily dose, is at least 225 mg/kg body weight (dry weight) and preferably at least 435 mg/kg body weight (dry weight).

In a further aspect of the invention the administration of the composition of the invention is once per two or three days up to at least once per day, preferably before and/or immediately after resistance exercise training, more preferably immediately after resistance exercise training, most preferably between 20 minutes and two hours after exercise.

In a further aspect of the invention there is provided a use of whey growth factor extract, isolated from a milk product by cation exchange chromatography, and an additional protein source for the production of a medicament for the treatment of a subject in need of improved skeletal muscle strength and/or size.

In a further aspect of the invention, there is provided a food or drink comprising the composition of the invention for use in a method to increase skeletal muscle strength and/or size in a subject undertaking resistance exercise training.

In yet a further aspect of the invention there is provided a use of the composition of the invention for the manufacture of a food or drink to increase skeletal muscle strength and/or size in a subject undertaking resistance exercise training.

### BRIEF DESCRIPTION OF THE DRAWINGS

FIG. 1 Cybex NORM dynamometer repetition maximum strength test results for leg press of adult subjects, at rest and performed at the start of the trial [pre-training] and following 6 and 12 weeks of treatment [post-training] with 20 g WPI (Group A), 1 g WGFE +20 g WPI (Group B1), or 2 g WGFE+20 g WPI (Group B2) administered immediately following 3 hours of resistance exercise training. Results are presented as mean±SEM.

FIG. 2 Percentage changes in fibre type composition in the vastus lateralis muscle of adult subjects, at 12 weeks of resistance exercise training and treatment [post-training] with 20g WPI (Group A), 1 g WGFE+20 g WPI (Group B1), or 2 g WGFE+20 g WPI (Group B2). Results are presented as mean±SEM.

FIG. 3 Fold change in Pax 7 and Syndecan 3 gene (mRNA) expression in the vastus lateralis muscle of adult subjects, at rest and following 3 hours of resistance exercise training performed at the start of the trial [pre-training] and following 12 weeks of treatment [post-training] with 20g WPI (Group A), 1 g WGFE+20 g WPI (Group B1), or 2 g WGFE+20 g WPI (Group B2). Results are presented as mean±SEM.

FIG. 4A: L6 myoblast growth assay over 48 h in response to stimulation with either LP (WGFE) or colostrum at concentrations ranging from 0.04-5 mg/ml. Each point represents the arithmetic mean±SEM of triplicate determinations. As a positive control, 10% FCS was included. Colostrum was produced by Murray Goulburn Co-Op Pty Ltd. 4B: L6 myoblast growth assay over 48 h, measuring dose response to LP (WGFE) up to 10 mg/ml. Each point represents the arithmetic mean±SEM of triplicate determinations. 4C: L6 myoblast growth assay over 48 h in response to LP (WGFE) at 5 mg/mi vs. unstimulated. The values plotted represent the arithmetic mean±SEM of the average response obtained in 3 independent experiments.

FIG. 5: BalbC 3T3 Fibroblast cell growth assay over 48 h in response to CPI (WPI) at 100 mg/ml or FMP (WGFE) at 100 mg/ml or other milk fractions such as colostrum, lactoferrin (LF), and 10% FCS (‘ve’ control). The values plotted represent the arithmetic mean±SEM of the average response obtained in 3 independent experiments.

### DETAILED DESCRIPTION OF THE INVENTION

The invention relates to a composition and method which enables persons undergoing resistance exercise training to increase their muscle strength.

It is thus an object of the present invention to provide a composition and method which is improved over the prior art for increasing muscle strength.

According to one aspect of the invention, there is provided a skeletal muscle strength-enhancing composition comprising whey growth factor extract isolated from a milk product by cation exchange chromatography.

The whey growth factor extract for use in the invention may be isolated from milk, skim milk, milk derivatives, whey, colostrum, and colostrum derivatives by, for example, the method described in Australian Patent No. 645589 (PCT/AU91/00303) which is incorporated herein by reference. This method essentially relies on strong cation exchange chromatography to selectively extract basic proteins from the starting material to constitute whey growth factor extract.

### Process for Producing WGFE Fraction

A preferred method of producing WGFE for use in the invention is to use a column packed with SP (sulphopropyl) Sepharose To the column a flow of a dairy product, preferably skimmed milk, is applied until the volume of milk applied is up to 1000 times the volume of the resin packed into the column. The milk remaining in the column is removed with a buffer of low ionic strength (<0.008M NaCl or equivalent) for 10 min. The WGFE fraction is eluted from the column with a buffer containing sodium ions equivalent to 0.4-0.5M NaCl (though other cations would be suitable), most preferably 0.4M NaCl.

The mobile phase may have a pH within a broad range, such as 4.5-9.0, preferably 5.5-7.5, most preferably about 6.5. At the upper and lower limits both protein stability and the ability of proteins to bind to the cation exchange resin become influenced. A pH in the range 5.5-7.5 provides the highest WGFE yields.

The type of cation exchange resin suitable for adsorption of the WGFE components may include resins such as Sepharose cation exchange resin beads. For example, SP Sepharose Big Beads and CM Sepharose beads (products of GE Healthcare) which contain sulfopropyl functional groups and carboxymethyl groups, respectively, are suitable. The size of the cation exchange resin beads is preferably in the range from 45-300 μm. Both SP Sepharose beads in the range 45-165 μm and in the range 100-300 μm are suitable for WGFE purification according to the invention.

One of the further treatments to which the WGFE fraction can be subjected is desalting by, for example, dialysis or ultrafiltration.

Accordingly, in a further aspect of the invention, there is provided a composition according to the above wherein the whey growth factor extract is isolated from whey or skim milk. The whey used as starting material may be cheese whey, rennet casein whey, acid casein whey, or concentrates thereof. The amounts of whey growth factor extract and protein source to use according to the invention are to be sufficient for an improved gain of muscle strength and/or size or therapeutic effect.

In another aspect of the invention there is provided a dosage regime wherein the amount of whey growth factor extract administered, per daily dose, is at least 5 mg/kg body weight to 12.5 mg/kg body weight. Preferably the daily dose of whey growth factor extract is at least 25 mg/kg body weight.

In a further aspect of the invention there is provided a method of improving skeletal muscle strength and/or size in subjects undertaking resistance exercise training comprising administering to the subjects an efficacious amount of a composition comprising whey growth factor extract.

When the composition includes an additional protein source, which may be any protein source suitable for consumption such as WPI, post-exercise muscle strength is increased compared to subjects administered a protein source alone. The protein source may be obtained from whole milk, preferably whey protein and more preferably whey protein isolate (WPI). One such whey protein isolate is available commercially under the trade name NatraPro™ by Murray Goulburn Co-Op Company Ltd. A typical composition of NatraPro™ WPI includes:

| | |
|---|---|
| Moisture | 5.0% |
| Fat | 0.5% |
| pH (5% solution) | 6.3 |
| Ash | 3.7% |
| Lactose | 0.5% |
| Protein (TN × 6.38) | 90.0% |
| Sodium | 0.7% |
| Phosphorous | 0.3% |
| Calcium | 0.15% |

Preferably the composition according to the invention also contains a protein source and, in a dosage regime, the amount of protein source per daily dose administered is at least 225 mg/kg body weight (dry weight) and preferably at least 435 mg/kg body weight (dry weight).

It will be apparent to those skilled in the art that the administration of such a composition may be on the days of training or on the days of training and/or other days, providing the regimen of administration results in increased muscle strength and/or size. Preferably administration is on the days of exercise and more preferably administration occurs either just before and/or after the exercise. More preferably, administration occurs between 20 minutes and 2 hours after exercise. Accordingly, in a preferred aspect of the invention, there is provided a method as described above wherein the administration is immediately after the exercise.

In a further aspect of the invention there is provided a use of whey growth factor extract for the production of a medicament for the treatment of subjects in need of increased skeletal muscle strength and/or size. For example, patients with muscle wasting, or the elderly, may require resistance exercise training to build up their muscle strength once more. A medicament comprising the composition of the invention may further assist in the subject's recovering their muscle strength. Moreover, the composition of the invention may be used to increase muscle strength in non-human mammals such as horses, greyhounds and others in which increased muscle strength is desirable.

In a further aspect of the invention, there is provided a food or drink comprising the composition of the invention for use in a method to increase skeletal muscle strength and/or size in subjects undertaking resistance exercise training.

In yet a further aspect of the invention there is provided a use of the composition of the invention for the manufacture of a food or drink to improve skeletal muscle strength and/or size in subjects undertaking resistance exercise training. It would be understood that the composition of the invention can be produced in the form of a tablet or capsule for administration to a subject undertaking resistance exercise training.

WPI typically contains about 90% w/v protein; therefore 20 g of WPI contains about 18 g w/v proteins as a nutritional source.

Whey growth factor extract typically contains about 85% w/w protein; therefore 2 g of whey growth factor extract contains about 1.7 g w/v proteins as a nutritional source.

It will be appreciated that the present invention described herein is not to be limited to specific examples of features disclosed.

### EXAMPLES

### Example 1

### Clinical Trial

A clinical trial was conducted in which 20 young males participated in a three month randomised double-blinded resistance training program. Whey growth factor extract was prepared according to the method broadly described in Australian Patent No. 645589 (PCT/AU91/00303), more specifically described supra. Each whey protein formulation contained an artificial sweetener (Nutrasweet™, Nutrasweet Company, USA). Whey protein formulations were consumed immediately after each exercise session, with each subject completing three supervised exercise sessions per week.

Subjects were randomly assigned to one of three supplement groups:

Group A: NatraPro (WPI); 20 g per dose; n=7

Group B1: NatraPro (WPI) plus WGFE; 20 g WPI plus 1 g WGFE per dose; n=6

Group B2 NatraPro (WPI) plus WGFE; 20 g WPI plus 2 g WGFE per dose; n=7

A typical composition of NatraPro™ WPI includes:

| | |
|---|---|
| Moisture | 5.0% |
| Fat | 0.5% |
| pH (5% solution) | 6.3 |
| Ash | 3.7% |
| Lactose | 0.5% |
| Protein (TN × 6.38) | 90.0% |
| Sodium | 0.7% |
| Phosphorous | 0.3% |
| Calcium | 0.15% |

A typical composition of WGFE according to the invention includes:

| | |
|---|---|
| Moisture | 5.0% |
| Fat | <0.5% |
| pH (5% solution) | 6.7 |
| Protein (TN × 6.38) | 95.0% |
| Ash | 1.5% |
| pH (2% solution) | 5.5-6.5 |

Muscular strength was analysed by testing leg extension strength using a cybex NORM dynamometer.

### Muscle Analysis

Muscle samples were collected from the vastus lateralis muscle of the right leg using the percutaneous needle biopsy technique. Excised muscle tissue was visually inspected, dissected free of any fat or connective tissue and blotted to remove excess blood and immediately frozen in liquid nitrogen for subsequent analysis. A portion of the muscle tissue was mounted in an aqueous mounting medium and frozen in isopentane cooled in liquid nitrogen for subsequent immunohistochemical analysis.

### RNA Extraction & Gene Expression Analysis

RNA was extracted from skeletal muscle samples using the ToTALLY RNA kit and reagents (Ambion Inc.) according to the manufacturer's instructions. Total RNA concentrations and quality were determined using the Agilent 2100 Bioanalyzer (Agilent Technologies, Inc.). Subsequently, RNA was reverse transcribed into cDNA using the AMV reverse transcriptase kit protocols and reagents (Promega). Analysis of gene expression was performed on the Applied Biosystems 7500 Real-Time PCR System using gene specific primers designed using Primer Express 2.0 software.

### Immunohistochemistry

Serial sections (10 μm) of each sample were mounted on microscope slides for analysis of myosin heavy chain fibre type. An immunochistochemical technique based on the fast and slow isoforms of myosin was used to examine fibre type distribution and muscle cross-sectional area based on the protocol of

Behan. Cellular localisation of proteins was performed using standard immunohistochemical techniques and antibodies raised against the proteins of interest.

### Results

Results are presented as mean±SEM and significance calculated by two-way ANOVA using Bonforoni post hoc tests. No significant differences in age, weight, height, or BMI values were observed both pre and post training.

TABLE 1
Subject Characteristics

| | NatraPro | NatraPro | NatraPro |
|---|---|---|---|
| Subject | WPI (A) | WPI (B 1g) | WPI (B 2g) |
| Characteristics | (n = 7) | (n = 6) | (n = 7) |
| Age | 20.4 ± 0.6 | 19.5 ± 0.6 | 19.0 ± 0.4 |
| Height | 182.7 ± 3.3 | 180 ± 5.2 | 182.3 ± 4.1 |
| Weight | | | |
| Pre Training | 79.9 ± 4.0 | 80.3 ± 4.6 | 79.3 ± 6.0 |
| Post | 80.1 ± 3.2 | 80.0 ± 4.7 | 80.9 ± 5.9 |
| Training | | | |
| BMI | | | |
| Pre Training | 24.5 ± 0.8 | 24.3 ± 1.0 | 23.9 ± 1.1 |
| Post | 24.07 ± 1.1 | 24.2 ± 1.0 | 24.4 ± 1.0 |
| Training | | | |

All subjects demonstrated improvements in skeletal muscle strength over the 12 weeks of strength training.

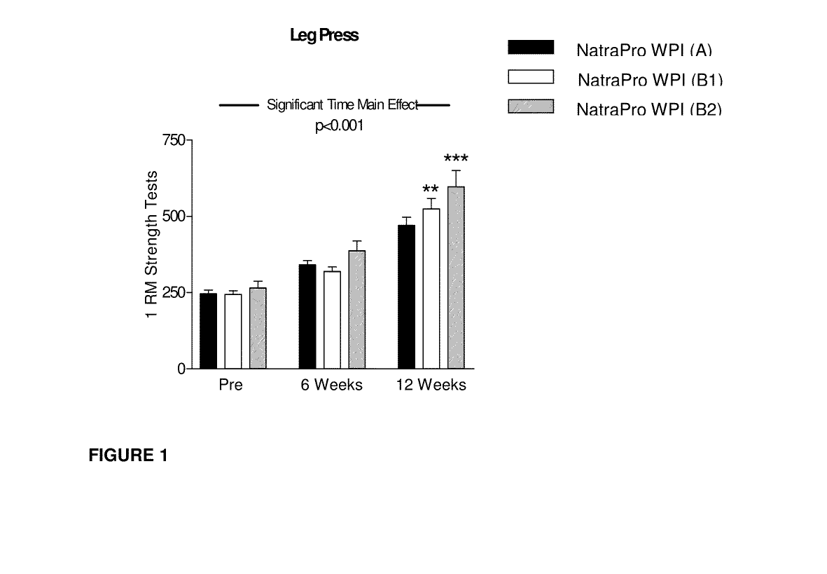

The NatraPro WPI (B1) group demonstrated an approximately 23% greater gain in leg press strength compared to group (A), whereas the NatraPro WPI (B2) group demonstrated a 35% greater gain in leg press strength compared to subjects receiving a protein source alone (NatraPro WPI (A), FIG. 1).

### Skeletal Muscle Fibre Type Changes

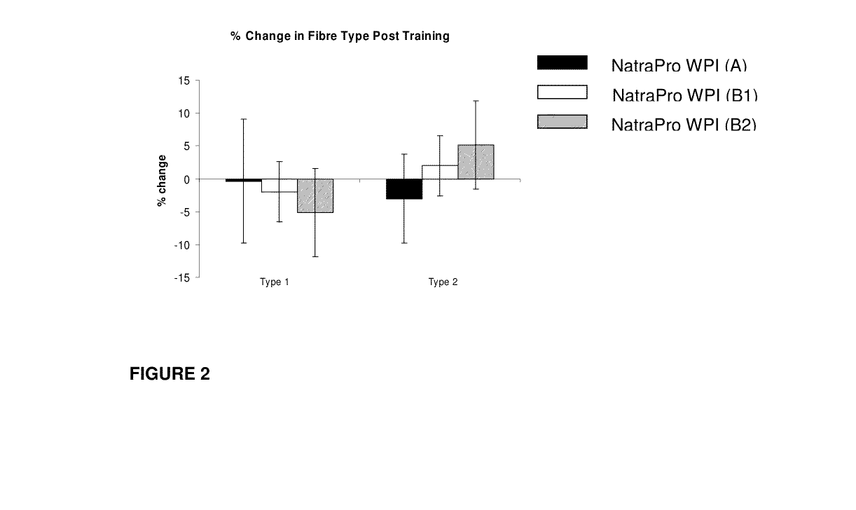

The percentage of muscle fibres classified as type 1 (slow/oxidative) or type 2 (fast/glycolytic) was altered by the administration of whey growth factor extract, which resulted in a trend towards an increase in the proportion of type 2B (most glycolytic) fibre types and a corresponding decrease in the proportion of type 1 (slow) fibre types (FIG. 2).

### Gene Expression Analysis

The increased expression and coordination of genes is an essential process activating stem cells that are located within the muscle bed (satellite cells) (Anderson & Wozniak, Can J Physiol Pharmacol. 2004; 82(5):300-10). Satellite cells are a population of adult stem cells that rapidly proliferate, before maturing and ultimately fusing with existing muscle fibres or join together to create new muscle fibres. Regulators of the activation of satellite cells include Syndecan-3 (a transmembrane heparin sulphate proteoglycan essential for satellite cell proliferation), Pax-7 (a protein of unknown function that is essential for satellite cell activation and necessary for muscle tissue repair) (Seale et al., Dev Biol. 2004; 15; 275(2): 287-300, Cornelison et al., Dev Biol. 2001; 239(1):79-94).

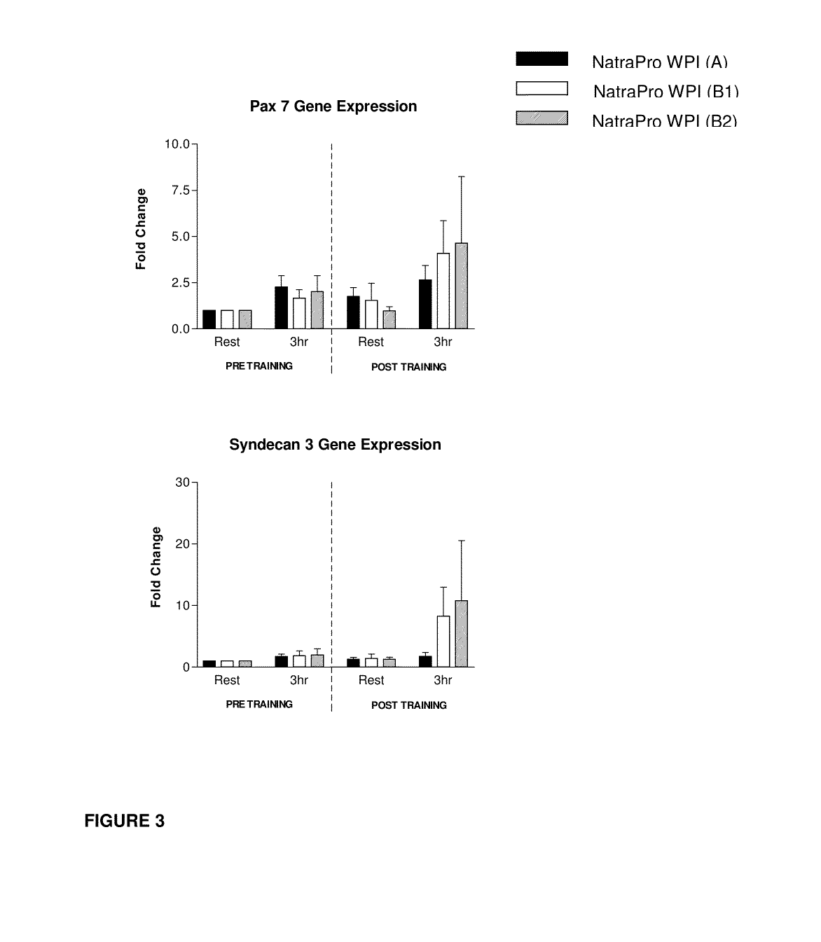

Pax 7 and Syndecan 3 expression tended to increase following 12 weeks of training and more so with administration of whey growth factor extract (FIG. 3).

The data supports that WGFE increases strength and/or size of muscle following resistance exercise training. The data also supports that WGFE in combination with an additional protein source such as WPI increases the strength gains to a greater extent than observed when WPI, a protein source known to be used by subjects undertaking resistance exercise training, is administered alone.

In support of this, it was observed that administration of whey growth factor extract promoted a switch in muscle fibre type from slow (type 1) to fast (type 2), and increased expression of muscle stem cell activation genes Pax 7 and Syndecan 3.

### Example 2

### Effect of WGFE on L6 Myoblast Cell Growth

An in vitro muscle cell growth study was conducted in which L6 myoblast cells were grown in the presence of WGFE (LP), colostrum, 10% fetal calf serum (FCS), or media only (unstimulated). Whey growth factor extract was prepared as for Example 1.

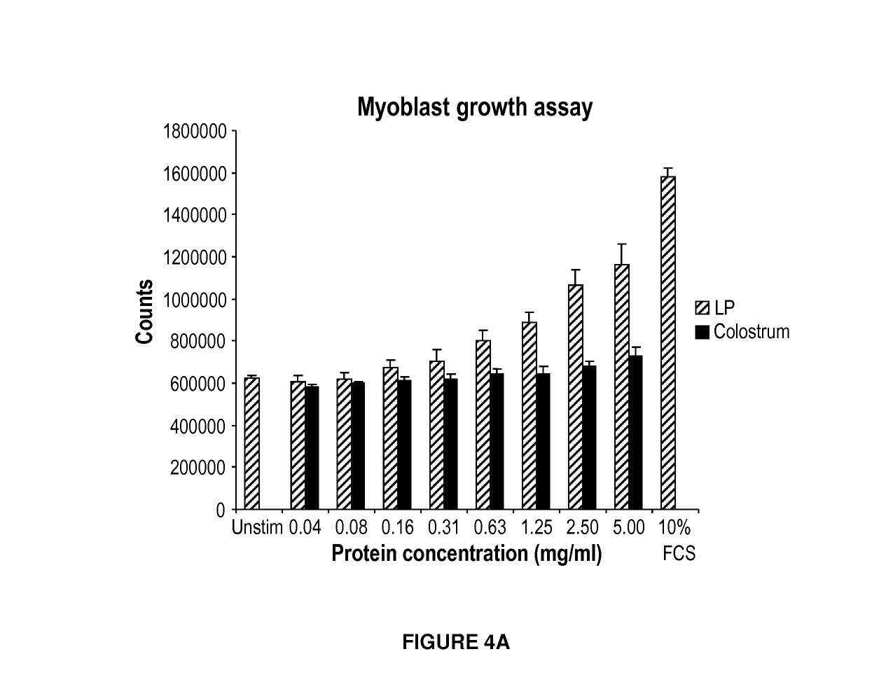

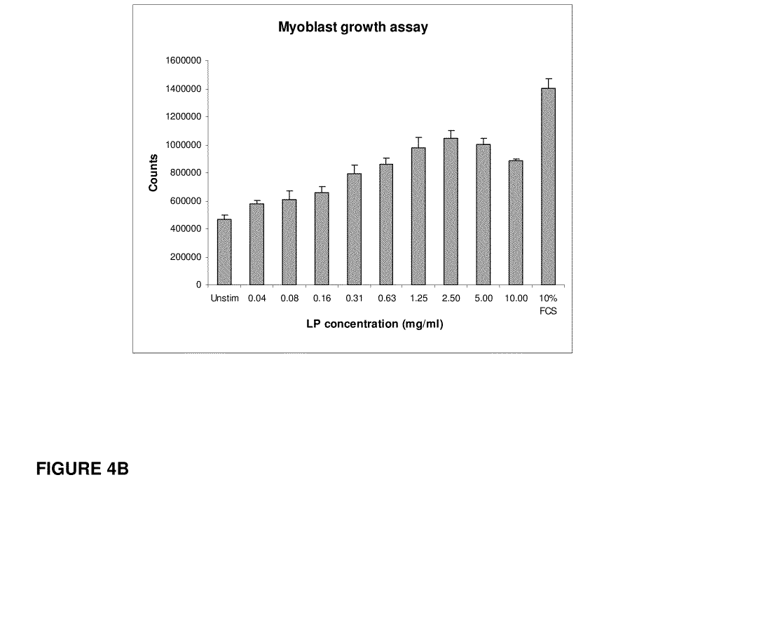

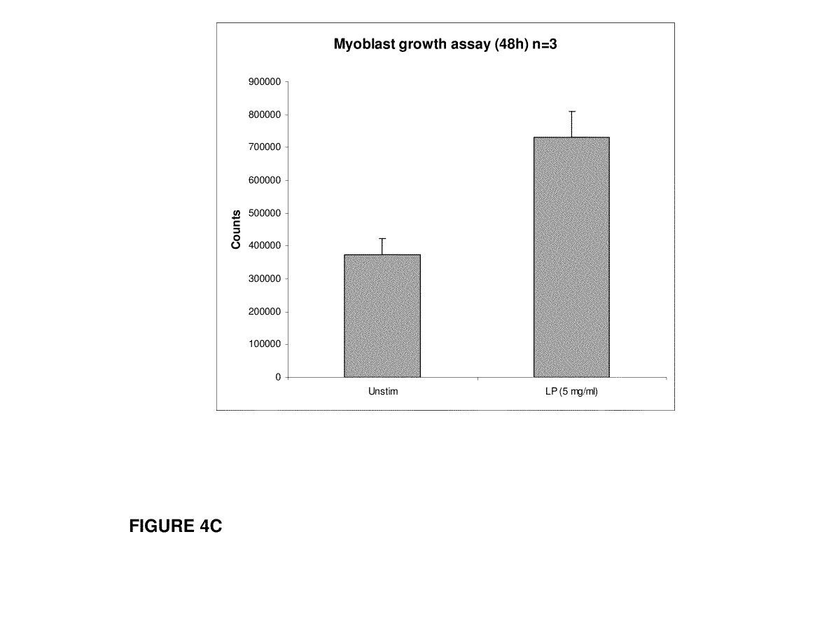

FIG. 4A: L6 myoblast growth assay over 48 h in response to stimulation with either WGFE or colostrum at concentrations ranging from 0.04-5 mg/ml. Each point represents the arithmetic mean±SEM of triplicate determinations.

There was approximately a 2 fold increase in cell number when stimulated with WGFE at 5 mg/ml over the 48 h. As a positive control, 10% FCS was included. Colostrum, produced by Murray Goulburn Co-Op Company Ltd, was also included for comparative purposes.

FIG. 4B: L6 myoblast growth assay over 48 h, measuring dose response to WGFE up to 10 mg/ml. Each point represents the arithmetic mean±SEM of triplicate determinations.

It appears that maximal response is achieved around 2.5-5 mg/ml, which then declines as the WGFE concentration increases to 10 mg/ml.

FIG. 4C: L6 myoblast growth assay over 48 h in response to WGFE at 5 mg/ml vs. unstimulated. The values plotted represent the arithmetic mean±SEM of the average response obtained in 3 independent experiments.

These data indicate that WGFE stimulates the growth of myoblast cells, whereas colostrum has very little stimulatory effect over the same concentration range, with an optimum stimulatory effect in the range 1.25-5.0 mg/ml. Moreover, the growth rate of myoblast cells treated with WGFE was about two fold higher than for untreated cells.

### Example 3

### Effect Of WGFE And WPI On Fibroblast Cell Growth

An in vitro fibroblast cell growth study was conducted in which BalbC 3T3 Fibroblast cells were grown in the presence of WGFE, WPI, colostrum, and other milk fractions, 10% FCS (a ‘+ve’ control), or media only Mill Whey growth factor extract was prepared as for Example 1. WPI is available commercially under the trade name NatraPro™ by Murray Goulburn Co-Op Company Ltd.

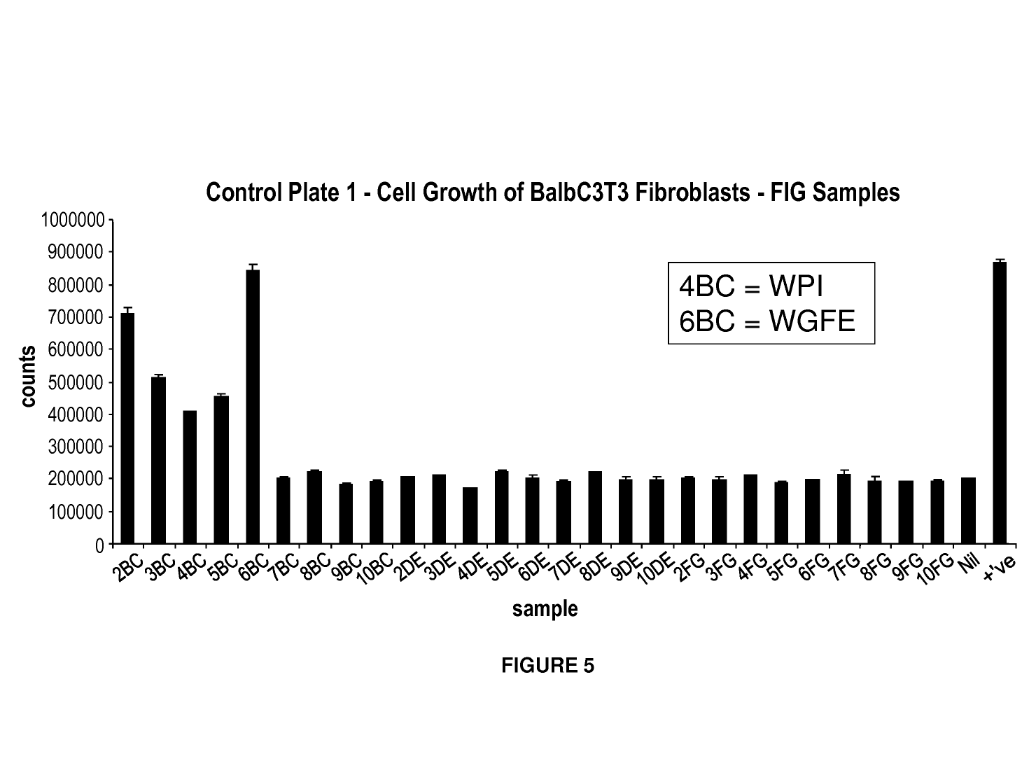

BalbC 3T3 Fibroblast cells were grown over 48 h in response to colostrum, WPI at 100 mg/ml, WGFE at 100 mg/ml, or various milk fractions as described above. The values plotted represent the arithmetic mean±SEM of the average response obtained in 3 independent experiments. This assay shows that WGFE is much more potent than WPI alone in stimulating cell growth (FIG. 5).

1-10. (canceled)

11. A method of increasing muscle strength and/or size in a subject undertaking resistance exercise training comprising administering to the subject once per two or three days up to at least once per day a composition comprising an effective amount of whey growth factor extract, isolated from a bovine milk product by a process comprising:
a) applying the milk product to a SP Sepharose cation exchange column,
b) washing the column with a buffer of low ionic strength,
c) eluting the whey growth factor extract fraction with a buffer containing in the range 0.4-0.5M NaCl, or equivalent ionic strength, at pH 6.5.

12. The method of claim 11, wherein the composition is administered in a daily dose of at least 5 mg/kg body weight whey growth factor extract.

13. The method of claim 12, wherein the composition comprises an additional protein source.

14. The method of claim 11, wherein the subject is administered an additional protein source taken separately.

15. The method of claim 13 or 14, wherein the subject is administered at least 225 mg/kg body weight of additional protein source.

16. The method of claim 15, wherein the additional protein source is whey protein.

17. (canceled)

18. The method of claim 11, wherein the administration to the subject is before, immediately after, or both before and immediately after resistance exercise training.

19. The method of claim 11, wherein the administration is immediately after resistance exercise training.

20. The method of claim 11, wherein the administration to the subject is between 20 minutes and two hours after resistance exercise training.

21. (canceled)

22. (canceled)

23. (canceled)

24. The method of claim 11, wherein the composition comprising an effective amount of whey growth factor extract is formulated as a food, drink, tablet or capsule.

25. The method of claim 24, wherein the composition is in the form of a nutritional bar or snack food.

26-29. (canceled)

30. The method of claim 11, wherein the whey growth factor extract has been isolated from a bovine milk product by a process comprising the steps:
a) applying skim milk to a SP Sepharose cation exchange column,
b) washing the column with a buffer of less than 0.008M NaCl,
c) eluting the whey growth factor extract fraction with a buffer containing 0.4M NaCl, at pH 6.5.

31. The method of claim 11, wherein the composition is administered in a daily dose of at least 12.5 mg/kg body weight whey growth factor extract.

32. The method of claim 11, wherein the composition is administered in a daily dose of at least 25 mg/kg body weight whey growth factor extract.
